(19) United States
(12) Patent Application Publication
(10) Pub. No.: US 2017/0124700 A1
(43) Pub. Date: May 4, 2017

(54) METHOD AND SYSTEM FOR MEASURING A VOLUME FROM AN ULTRASOUND IMAGE

(71) Applicant: General Electric Company, Schenectady, NY (US)

(72) Inventors: Subin Sundaran Baby Sarojam, Bangalore (IN); Pavan Kumar V. Annangi, Bangalore (IN); Tricia Eshelman, Wauwatosa, WI (US); Anders Herman Torp, Oslo (NO)

(21) Appl. No.: 14/928,241

(22) Filed: Oct. 30, 2015

(51) Int. Cl. G06T 7/00; G06T 7/60; G06F 3/0484; A61B 8/08

(52) U.S. Cl. CPC G06T 7/0012; A61B 8/0833; A61B 8/5207; A61B 8/523; A61B 8/5223; G06T 7/602; G06T 7/0085; G06F 3/0484; G06T 2207/10132

(57) ABSTRACT

Methods and systems for measuring a volume of an organ of interest (OOI) from an ultrasound image are provided. The methods and systems acquire a first ultrasound image of an OOI at a first plane and a second ultrasound image of the OOI at a second plane, and identify the OOI within the first ultrasound image and the second ultrasound image by executing a classification model stored on a memory. The methods and systems further determine a first boundary of the OOI within the first ultrasound image and a second boundary of the OOI within the second ultrasound image by executing a contour model stored in the memory, determine a first second dimensional length of the OOI from the first boundary and a third dimensional length of the OOI from the second boundary, and calculate a volume of the OOI derived from the first, second, and third dimensional links.

### BACKGROUND OF THE INVENTION

The subject matter disclosed herein relates generally to ultrasound imaging systems, and more particularly, to a method and apparatus for performing volume measurements using a mobile ultrasound imaging system of an organ of interest.

Ultrasound imaging systems typically include ultrasound scanning devices, such as, ultrasound probes having different transducers that allow for performing various different ultrasound scans (e.g., different imaging of a volume or body). Mobile or pocket sized ultrasound imaging systems are gaining significance due to their portability, low costs, and image quality. Mobile ultrasound imaging systems may be utilized to perform various procedures that were once only accomplished in a dedicated medical facility, for example, a hospital. Mobile ultrasound imaging systems can include diagnostic tools based on acquired ultrasound images of the ultrasound imaging system. Some diagnostic tools can determine a volume of an organ of interest by the clinician, such as the bladder. The volume of an organ of interest can be used to diagnose a number of clinical conditions requiring treatment. For example, the differences between a pre-void and post-void volume of the bladder may be used for a urinary retention diagnosis.

However, currently available volume diagnostic tools for mobile ultrasound imaging system use either manual or automatic volume measurements. Manual volume measurements are time consuming requiring the clinician to identify edges and dimensions of the organ of interest from one or more ultrasound images. Alternatively, currently available automatic volume measurements need not be accurate. Further, due to the small screens and limited space for user interface components, conventional mobile ultrasound imaging systems do not provide assistance for protocol or step guidance for volume diagnostic tools. Thus, there is a need for a mobile ultrasound imaging system having automated end to end applications with various assistance tools, aiding the clinician to perform a volume measurement with minimal manual intervention.

### BRIEF DESCRIPTION OF THE INVENTION

In one embodiment, a method of using a mobile ultrasound imaging system to determine a volume of an organ of interest (OOI) from a plurality of ultrasound images is provided. The method may include acquiring a first ultrasound image of an OOI at a first plane and a second ultrasound image of the OOI at a second plane. The first plane be orthogonal to the second plane. The method may also include using one or more processors to identify the OOI within the first ultrasound image and the second ultrasound image by executing a classification model stored in memory. The classification model includes a feature vector corresponding to the OOI. The method may also include using the one or more processors to determine a first boundary of the OOI within the first ultrasound image and a second boundary of the OOI within the second ultrasound image by executing a contour model stored in the memory. The method may also include determining first and second dimensional lengths of the OOI from the first boundary and a third dimensional length of the OOI from the second boundary, and calculating a volume of the OI tried the first, second, and third dimensional lengths.

In another embodiment, a mobile ultrasound imaging system is provided. The mobile ultrasound imaging system may include a portable host system having one or more processors and memory for storing a plurality of applications for execution of programmed instructions. When a select application is activated, the one or more processors perform a plurality of operations. The operations may include acquiring a first ultrasound image of an organ of interest (OOI) at a first plane and a second ultrasound image of the OOI at a second plane. The first plane being orthogonal to the second plane. The operations may further include identifying the OOI within the first ultrasound image and the second ultrasound image by executing a classification model stored in memory. The classification model may have a feature vector corresponding to the OOI. The operations may also include determining a first boundary of the OOI within the first ultrasound image and a second boundary of the OOI within the second ultrasound image by executing a contour model stored in the memory. The operations may further include determining first and second dimensional lengths of the OOI from the first boundary and a third dimensional length of the OOI from the second boundary, and calculating a volume of the OOI derived from the first, second, and third dimensional lengths.

In another embodiment, a tangible and non-transitory computer readable medium include one or more programmed instructions configured to direct one or more processors. The one or more programmed instructions may be configured to direct the one of more processors to acquire a first ultrasound image of an organ of interest (OOI) at a first plane and a second ultrasound image of the OOI at a second plane. The first plane being orthogonal to the second plane. The one or more programmed instructions may also be configured to direct the one or more processors to identify the OOI within the first ultrasound image and the second ultrasound image by executing a classification model stored in memory. The classification model includes a feature vector corresponding to the OOI. The one or more programmed instructions may also be configured to direct the one of more processors to determine a first boundary of the OOI within the first ultrasound image and a second boundary of the OOI within the second ultrasound image by executing a contour model stored in the memory, and determine first and second dimensional lengths of the OOI from the first boundary and a third dimensional length of the OOI from the second boundary. The one or more programmed instructions may also be configured to direct the one or more processors to calculate a volume of the OOI derived from the first, second, and third dimensional lengths.

### BRIEF DESCRIPTION OF THE DRAWINGS

FIG. 1 illustrates an exemplary mobile ultrasound imaging system formed, in accordance with various embodiments described herein.

FIG. 2 is a system block diagram of the mobile ultrasound imaging system shown in FIG. 1.

FIG. 3 is a screen shot of a graphical user interface shown on a touchscreen display of a host system shown in FIG. 1, in accordance with various embodiments described herein.

FIG. 4 is a screen shot of a graphical user interface on the touchscreen display of a host system shown in FIG. 1, in accordance with various embodiments described herein.

FIG. 5 is a block diagram of an ultrasound processor module of a host system shown in FIG. 1, in accordance with various embodiments described herein.

FIG. 6 is a screen shot of the graphical user interface of FIG. 4 with a diagnostic and tool menu, in accordance with various embodiments described herein.

FIG. 7 illustrates a simplified flowchart illustrating a method of using a mobile ultrasound imaging system to determine a volume of an organ of interest, in accordance with various embodiments described herein.

FIG. 8 is a screen shot of a graphical user interface, in accordance with various embodiments described herein.

FIG. 9 is an illustration of orthogonal planes of an organ of interest, in accordance with various embodiments described herein.

FIG. 10A is a workflow diagram illustrating operational steps of a controller executing a classification model, in accordance with various embodiments described herein.

FIG. 10B illustrates an ultrasound image being adjusted during the workflow diagram of FIG. 10A and a contour model, in accordance with various embodiments described herein.

FIG. 11 is a screen shot of graphical user interface, in accordance with various embodiments described herein.

FIG. 12 is a screen shot of graphical user interface, in accordance with various embodiments described herein.

FIG. 13A is a screen shot of graphical user interface, in accordance with various embodiments described herein.

FIG. 13B is a screen shot of graphical user interface, in accordance with various embodiments described herein.

### DETAILED DESCRIPTION OF THE INVENTION

The foregoing summary, as well as the following detailed description of certain embodiments, will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors, controllers or memories) may be implemented in a single piece of hardware (e.g., a general purpose signal processor or random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

Also as used herein, the phrase “generating an image” is not intended to exclude embodiments of the present invention in which data representing an image is generated, but a viewable image is not. Therefore, as used herein the term “image” broadly refers to both viewable images and data representing a viewable image. However, many embodiments generate, or are configured to generate, at least one viewable image.

Described herein are various embodiments for a mobile ultrasound imaging system utilizing an assisted volume technique for faster and accurate volume measurements of an organ of interest, such as a bladder. The mobile ultrasound imaging system may include a portable host system that executes an automatic and/or semi-automatic method for calculating a volume from two orthogonal two dimensional (2D) ultrasound images of an organ of interest (OOI). Various embodiments of the mobile ultrasound imaging system may provide assistance to the clinician in terms of defining the steps to acquire orthogonal 2D ultrasound images of the OOI, corresponding to a sagittal plane and a transverse plane of the OOI. The mobile ultrasound imaging system may automatically execute a segmentation algorithm that includes a classification model and a contour model, which are used to determine a boundary or contour of the OOI within each 2D ultrasound image of the OUI and determine dimensions (e.g., length, width and height) of the OOI.

Additionally or alternatively, the mobile ultrasound imaging system may display the ultrasound images with an overlay of the contour with calipers positioned at the widest points of the OOI corresponding to a dimension of the OOI. Optionally, the clinician may adjust the calipers and thereby the dimensional measurements of the OOI using a graphical user interface of the mobile ultrasound imaging system. By adjusting the calipers, the accuracy of the dimensional measurements are increased. Based on the dimensions of the OOI, the mobile ultrasound imaging system calculates a volume of the OOI. For example, the volume is estimated by an ellipsoid approximation.

A technical effect of at least one embodiment includes fast identification of the OOI. A technical effect of at least one embodiment includes increased accuracy of dimensional measurements of the OOI.

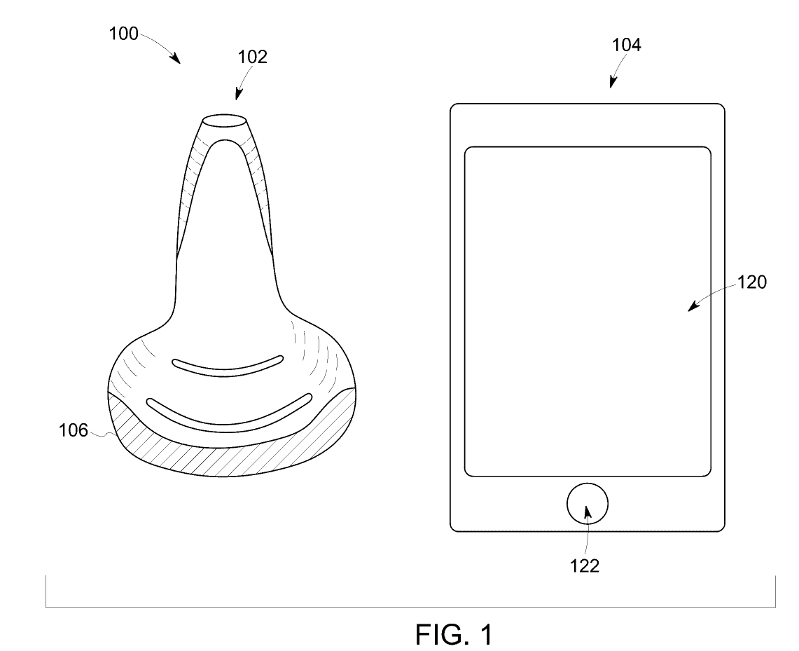

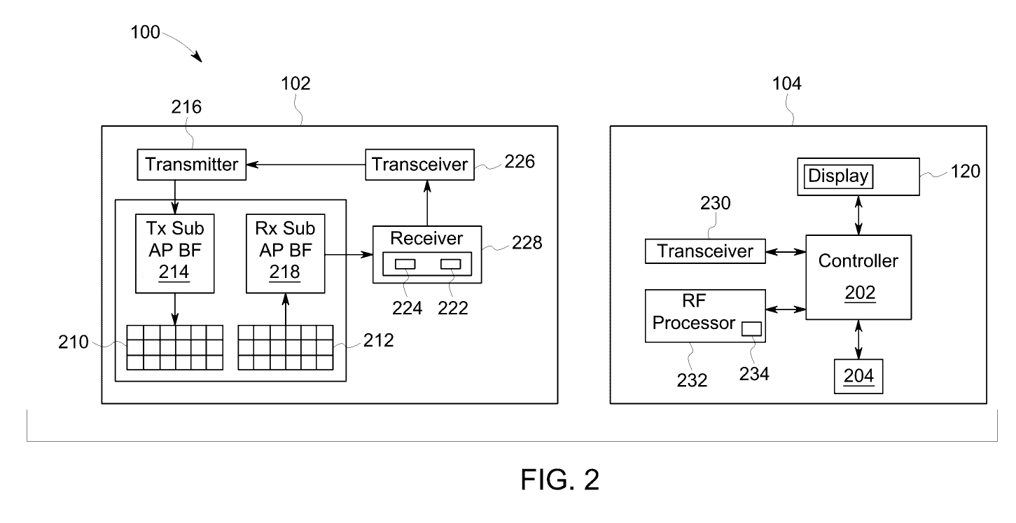

Various embodiments described herein may be implemented as a mobile ultrasound imaging system 100 as shown in FIG. 1. More specifically, FIG. 1 illustrates an exemplary mobile ultrasound imaging system 100 that is constructed in accordance with various embodiments. The ultrasound imaging system 100 includes a portable host system 104. The portable host system 104 may be a portable hand-held device, for example, a mobile phone such as a smart phone, a tablet computer, and/or the like. The portable host system 104 may support one or more applications that are executed by a controller 202, shown in FIG. 2, of the portable host system 104.

An application may correspond to one or more software modules stored on a memory 204 that when executed have the controller 202 perform one or more coordinated functions, tasks, and/or activities. One or more applications may correspond to medical imaging functions such as an ultrasound imaging application, medical diagnostic tools (e.g., organ volume), and/or the like. Additionally or alternatively, one or more applications may correspond to non-medical imaging functions (e.g., not using or based on ultrasound data) such as a word processing application, a disc authoring application, a gaming application, a telephone application, an e-mail application, an instant messaging application, a photo management application, a digital camera application, a web browsing application, a GPS mapping application, a digital music player application, a digital video player application, and/or the like. Optionally, one or more of the applications may be received by the portable host system 104 remotely. The one or more applications may be executed on the portable host system 104, and use a common physical user interface, such as a touchscreen display 120 (e.g., a touch-sensitive display) or one or more tactile buttons 122.

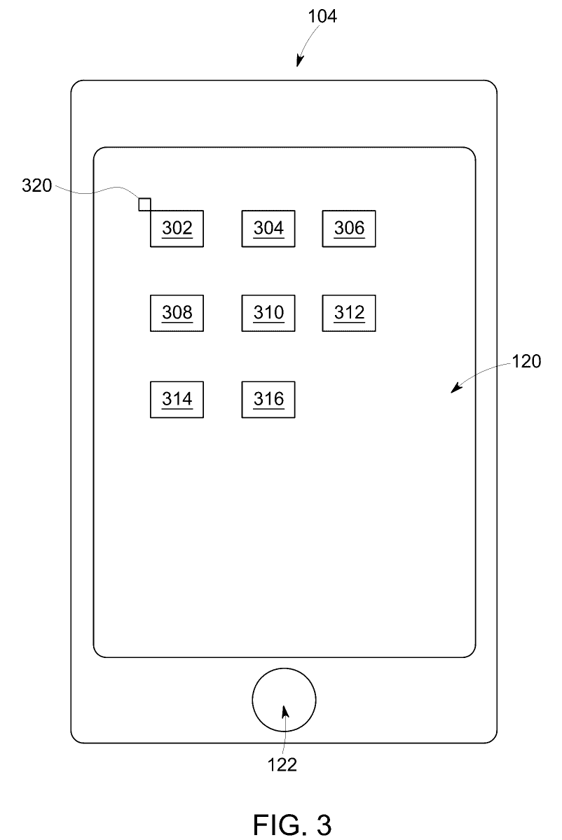

For example, the touchscreen display 120 may display information corresponding to one or more user selectable icons 302-316 (shown in FIG. 3) of a graphical user interface (GUI). One or more functions of the touchscreen display 120 as well as the corresponding information displayed on the device may be adjusted and/or varied from one application to the next and/or within a respective application.

The ultrasound imaging system 100 may include an ultrasound probe 102. The ultrasound probe 102 includes a transducer array 100, such as a phased array having electronics to perform sub-aperture (SAP) beamforming. For example, transducer array 100 may include piezoelectric crystals that emit pulsed ultrasonic signals into a body (e.g., patient) or volume. The ultrasonic signals may include, for example, one of more reference pulses, one or more pushing pulses (e.g., sheer-waves), and/or one or more tracking pulses. At least a portion of the pulsed ultrasonic signals are back-scattered from structures in and around the OOI and measured by the ultrasound probe 102. The ultrasound probe 102 may be connected wirelessly or with a cable to the host system 104. In one embodiment, the ultrasound probe 104 may be a universal probe which integrates both a phased array transducer and a linear transducer into the same probe housing.

In various embodiments, the ultrasound probe 102 may include an analog front end (AFE) 220, shown in FIG. 2, which may include built-in electronics that enable the ultrasound probe 102 to transmit digital signals to the portable host system 104. The portable host system 104 then utilizes the digital signals to reconstruct an ultrasound image based on the information received from the ultrasound probe 102.

FIG. 2 is a schematic block diagram of the imaging system 100 shown in FIG. 1. In various embodiments, the ultrasound probe 102 includes a two-dimensional (2D) array 200 of elements. The ultrasound probe 102 may also be embodied as a 1.25 D array, a 1.5 D array, a 1.75 D array, a 2D array, and/or the like. Optionally, the ultrasound probe 102 may be a stand-alone continuous wave (CW) probe with a single transmit element and a single receive element. In various embodiments, the ultrasound probe 102 includes a transmit group of elements 210 and a receive group of elements 212. A sub-aperture transmit beamformer 214 controls a transmitter 216 which, through transmit sub-aperture beamformers 214, drives the group of transmit elements 210 to emit, for example, CW ultrasonic transmit signals into a region of interest (e.g., human, animal, underground cavity, physical structure and the like). The transmitted CW ultrasonic signals are back-scattered from structures in and around the OOI, like blood cells, to produce echoes which return to the receive group of elements 212. The receive group of elements 212 convert the received echoes into analog signals as described in more detail below. A sub-aperture receive beamformer 218 partially beamforms the signals received from the receive group of elements 212 and then passes the partially beamformed signals to a receiver 228.

The sub-aperture transmit beamformer 214 may be configured to reduce a number of system channels utilized to process signals from the large number of transducer elements 210. For example, assume that there are m elements 210. In various embodiments, m channels are then utilized to couple the m elements 210 to the sub-aperture beamformer 214. The sub-aperture beamformer 214 then functions such that n channels of information are passed between the transmitter 216 and the sub-aperture beamformer 214, wherein n<m. Moreover, assume that there are m elements 212. In various embodiments, m channels are then utilized to couple the m elements 212 to the sub-aperture beamformer 218. The sub-aperture beamformer 218 then functions such that n channels of information are passed between the receiver 228 and the sub-aperture beamformer 218, wherein n<m. Thus, the sub-aperture beamformers 214 and 218 function to output fewer channels of information than are received from the elements 210 and 104.

In various embodiments, the receiver 228 may include the AFE 220. The AFE 220 may include for example, a plurality of demodulators 224 and a plurality of analog/digital (A/D) converters 222. In operation, the complex demodulators 224 demodulate the RF signal to form IQ data pairs representative of the echo signals. The I and Q values of the beams represent in-phase and quadrature components of a magnitude of echo signals. More specifically, the complex demodulators 224 perform digital demodulation, and optionally filtering as described in more detail herein. The demodulated (or down-sampled) ultrasound data may then be converted to digital data using the A/D converters 222. The A/D converters 222 convert the analog outputs from the complex demodulators 224 to digital signals that are then transmitted to the portable host system 104 via a transceiver 226. In various embodiments, the transceiver 226 is configured to wirelessly transmit and/or receive digital information (e.g., ultrasound data) based on a wireless protocol from the portable host system 104. For example, the wireless protocol may be Bluetooth, Bluetooth low energy, ZigBee, and/or the like. In other embodiments, the ultrasound probe 102 may be physically coupled to the portable host system 104 via a cable.

The beamformers 214 and 218, and the complex demodulators 224 facilitate reducing the quantity of information that is transmitted from the ultrasound probe 102 to the portable host system 104. Accordingly, the quantity of information being processed by the portable host system 104 is reduced and ultrasound images of the patient may be generated, by the portable host system 104, in real-time as the information is being acquired from the ultrasound probe 102.

The portable host system 104 may include a controller 202 operably coupled to the memory 204, the touchscreen display 120, and the transceiver 230. The controller 202 may include one or more processors. Additionally or alternatively, the controller 202 may include a central controller circuit (CPU), one or more microprocessors, a graphics controller circuit (GPU), or any other electronic component capable of processing inputted data according to specific logical instructions. The controller 202 may execute programmed instructions stored on a tangible and non-transitory computer readable medium (e.g., memory 204, integrated memory of the controller 202 such as EEPROM, ROM, or RAM) corresponding to one or more applications. For example, when a select application is activated by the user, the controller 202 executes the programmed instructions of the select application.

The transceiver 230 may include hardware, such as a processor, controller, or other logic based device to transmit, detect and/or decode wireless data received by an antenna (not shown) of the transceiver 230 based on a wireless protocol (e.g., Bluetooth, Bluetooth low energy, ZigBee, and/or the like). For example, the transceiver 230 may transmit to and/or receive wireless data that includes ultrasound data from the transceiver 226 of the ultrasound probe 102.

In various embodiments, the host system 104 may include hardware components, including the controller 202, that are integration to form a single “System-On-Chip” (SOC). The SOC device may include multiple CPU cores and at least one GPU core. The SOC may be an integrated circuit (IC) such that all components of the SOC are on a single chip substrate (e.g., a single silicon die, a chip). For example, the SOC may have the memory 204, the controller 202, the transceiver 230 embedded on a single die contained within a single chip package (e.g., QFN, TQFP, SOIC, BGA, and/or the like).

The touchscreen display 120 may include a liquid crystal display, an organic light emitting diode display, and/or the like overlaid with a sensor substrate (not shown). The sensor substrate may include a transparent and/or optically transparent conducting surface, such as indium tin oxide (ITO), a metal mesh (e.g., a silver nano-tube mesh, and carbon match, a graph feed mesh), and/or the like. The sensor substrate may be configured as an array of electrically distinct rows and columns of electrodes that extend through a surface area of the touchscreen display 120. The sensor substrate may be coupled to a touchscreen controller circuit (not shown).

A touchscreen controller circuit may include hardware, such as a processor, a controller, or other logic-based devices and/or a combination of hardware and software which is used to determine a position on the touchscreen display 120 activated and/or contacted by the user (e.g., finger(s) in contact with the touchscreen display 120). In various embodiments, the touchscreen controller circuit may be a part of and/or integrated with the controller 202. The touchscreen controller may determine a user select position activated and/or contacted by the user by measuring a capacitance for each electrode (e.g., self-capacitance) of the sensor substrate.

For example, the touchscreen controller may transmit a current drive signal along a single electrode and measure a capacitance along the single electrode. Additionally or alternatively, the touchscreen controller may measure a capacitance for each intersection of a row and column electrode (e.g., mutual capacitance). For example, the touchscreen controller may transmit a current drive signal along a first electrode (e.g., a row electrode, a column electrode) and measure a mutual capacitance from a second electrode (e.g., a column electrode, a row electrode). Based on the measured capacitance, the touchscreen controller may determine whether a finger(s) from the user is in contact and/or proximate to the sensor substrate. For example, when the capacitance, of the single electrode or intersection, is above a predetermined threshold the touchscreen controller may determine that the user is activating the corresponding single electrode or intersection. Further, based on a location of the corresponding single electrode or intersection, the touchscreen controller may determine a position of the finger with respect to the touchscreen display 120. In another example, when the capacitance is below a predetermine threshold the touchscreen controller may determine that the single electrode or intersection is not activated. The touchscreen controller may output the user select position of the user input to the controller 202. In connection with FIG. 3, the controller 202 may determine activation of a select application based on the user select position of the contact by the user.

FIG. 3 is a screen shot of a GUI shown on the touchscreen display 120. The GUI may include one or more interface components, such as one or more user selectable icons 302-316 illustrated in FIG. 3. The interface components correspond to user selectable elements shown visually on the touchscreen display 120, and may be selected, manipulated, and/or activated by the user operating the touchscreen display 120. The interface components may be presented in varying shapes and colors. Optionally, the interface components may include text or symbols.

It should be noted that the layout of the icons 302-316 is merely for illustration and different layouts may be provided. Each of the one or more user selectable icons 302-316 may correspond to an application stored in the memory 204 and executable by the controller 202. In various embodiments, the icons 302-316 may include, for example, an ultrasound imaging application 302, a web browser application 304, an e-mail application 306, a GPS mapping application 306, a telephone application 308, a word processing application 310, a digital music player application 312, a digital video application 314, a digital camera application 316, and various other icons. The user selectable icons 302-316 may be any graphical and/or text based selectable element. For example, the icon 302 may be shown as an image of an ultrasound probe.

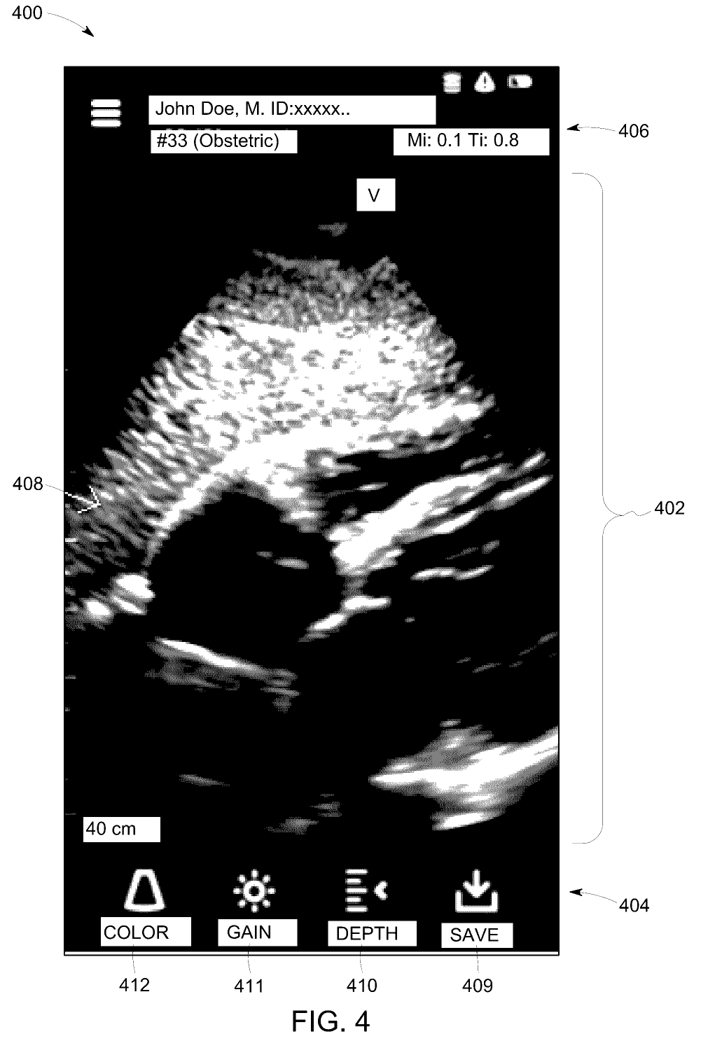

The controller 202 may determine when one of the selectable icons 302-316 and corresponding application is selected by the user select position determined from the touchscreen controller is approximately the same and/or within a predetermined distance of a position of a corresponding icon 302-316. For example, the controller 202 may receive a user select position 320 from the touchscreen controller. Since the user select position 320 is adjacent to or overlaid with the ultrasound imaging application 302, the controller 202 may determine that the ultrasound imaging application 302 is selected by the user. When selected, the controller 202 may execute the programmed instructions corresponding to the selected icon 302-316. For example, in connection with FIG. 4, when the ultrasound imaging application 302 is selected, the controller 202 may execute programmed instructions corresponding to the ultrasound imaging application.

FIG. 4 illustrates a GUI 400 displayed on the touchscreen display 120 of the host system 104 when the ultrasound imaging application is selected based on the programmed instructions corresponding to the ultrasound imaging application. The GUI 400 may include one or more interface components (e.g., menu bar 404, title bar 406) and an activity window 402.

The activity window 402 may correspond to an area of the GUI 400 for viewing results or outcomes of one or more operations performed by the controller 202. For example, the activity window 402 may include one or more ultrasound images 408, ultrasound videos, measurements, diagnostic results, data entry (e.g., patient information), and/or the like. It should be noted in various other embodiments the activity window 402 may be larger or smaller relative to the one or more interface components as illustrated in FIG. 4. Optionally the activity window 402 may be in a full-screen mode. For example, a size of the activity window 402 may encompass the touchscreen display 120.

The title bar 406 may identify information of the patient, user information, data and/or time information, and/or the like during operation of the ultrasound imaging application.

The menu bar 404 may correspond to a list of textual or graphical user selectable elements from which the user may select. For example, the menu bar 404 may include one or more icons 409-412 that correspond to one or more operations or functions that may be performed by the controller 202 when selected by the user.

For example, when the controller 202 executes programmed instructions corresponding to the ultrasound imaging application, the controller 202 may start acquiring ultrasound data from the ultrasound probe 102 and generate ultrasound images, as described above. Additionally or alternatively, the user may select one of the icons 410-412 to begin and/or adjust acquisition settings for the acquisition of the ultrasound images (e.g., adjust a gain, B-mode acquisition, color flow), select the icon 409 to save ultrasound images displayed in the activity window 402 to be used for diagnostic or measurement tools (e.g., measuring a volume of an OOI) by the controller 202, and/or the like. The programmed instructions for the one or more icons 409-412 (e.g., to acquire ultrasound images) may be included within the programmed instructions of the ultrasound imaging application stored in the memory 204, which includes algorithms for beamforming as well as subsequent signal and image processing steps utilized to process (e.g., an RF processor 232) and display the ultrasound information received from the ultrasound probe 102. In operation, the algorithms stored in the memory 204 may be dynamically configured or adjusted by the controller 202 according to a probe/application as well as the computing and/or power supply capabilities of the host system 104. The controller 202 may execute the beamforming algorithm stored in the memory 204 to perform additional or final beamforming to the digital ultrasound information received from the ultrasound probe 102, and outputs a radio frequency (RF) signal. Additionally or alternatively, the portable host system 104 may include a receive beamformer (not shown), which receives the digital ultrasound information and performs the additional or final beamforming.

The RF signal is then provided to an RF processor 232 that processes the RF signal. The RF processor 232 may include a complex demodulator 232 that demodulates the RF signal to form IQ data pairs representative of the echo signals, and one or more processors. The RF or IQ signal data may then be provided directly to the memory 204 for storage (e.g., temporary storage). Optionally, the output of the RF processors 232 may be passed directly to the controller 202. Additionally or alternatively, the RF processor 232 may be integrated with the controller 202 corresponding to programmed instructions of the ultrasound imaging application stored in the memory 204.

The controller 202 may further process the output of the RF processor 232 and prepare frames of ultrasound information for display on the touchscreen display 120. In operation, the controller 202 is configured to perform one or more processing operations according to a plurality of selectable ultrasound modalities on the acquired ultrasound data.

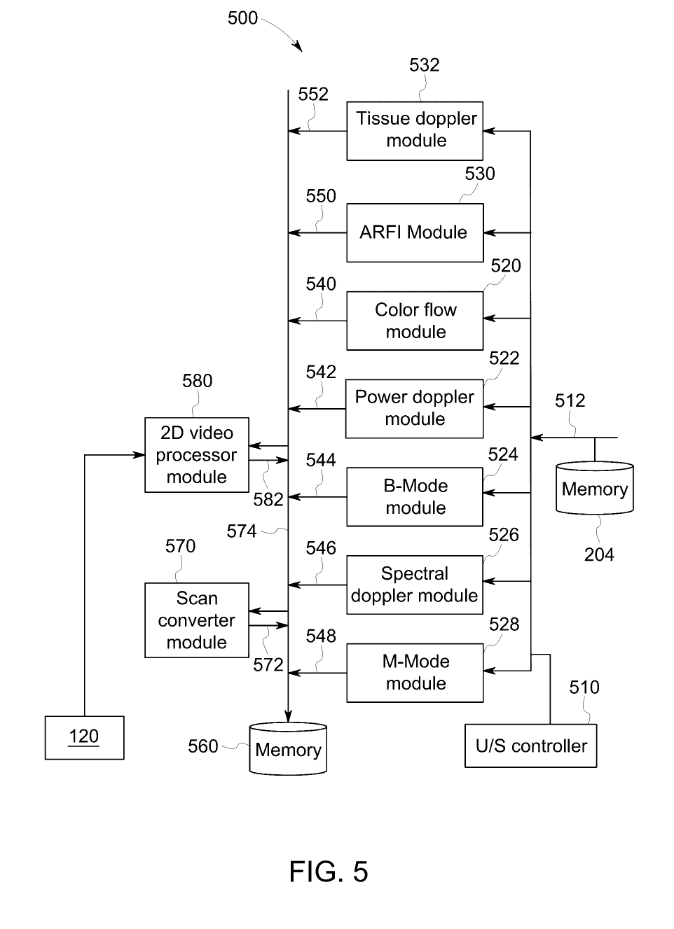

FIG. 5 illustrates an exemplary block diagram of an ultrasound processor module 500, which may be embodied in the controller 202 of FIG. 2 or a portion thereof. The ultrasound processor module 500 is illustrated conceptually as a collection of sub-modules corresponding to operations that may be performed by the controller 202 when executing programmed instructions for acquiring ultrasound images. Optionally, the one or more sub-modules may be implemented utilizing any combination of dedicated hardware boards, DSPs, processors, and/or the like of the host system 104. Additionally or alternatively, the sub-modules of FIG. 5 may be implemented utilizing one or more processors, with the functional operations distributed between the processors, for example also including a Graphics Processor Unit (GPU). As a further option, the sub-modules of FIG. 5 may be implemented utilizing a hybrid configuration in which certain modular functions are performed utilizing dedicated hardware, while the remaining modular functions are performed utilizing a processor. The sub-modules also may be implemented as software modules within a processing unit.

The operations of the sub-modules illustrated in FIG. 5 may be controlled by a local ultrasound controller 510 or by the controller 202. The controller 202 may receive ultrasound data 512 in one of several forms. In the exemplary embodiment of FIG. 2, the received ultrasound data 512 constitutes IQ data pairs representing the real and imaginary components associated with each data sample. The IQ data pairs are provided to one or more of a color-flow sub-module 520, a power Doppler sub-module 522, a B-mode sub-module 524, a spectral Doppler sub-module 526 and an M-mode sub-module 528. Optionally, other sub-modules may be included such as an Acoustic Radiation Force Impulse (ARFI) sub-module 530 and a Tissue Doppler (TDE) sub-module 532, among others.

Each of sub-modules 520-532 are configured to process the IQ data pairs in a corresponding manner to generate color-flow data 540, power Doppler data 542, B-mode data 544, spectral Doppler data 546, M-mode data 548, ARFI data 550, and tissue Doppler data 552, all of which may be stored in a memory 560 (or memory 204 shown in FIG. 2) temporarily before subsequent processing. For example, the B-mode sub-module 524 may generate B-mode data 544 including a plurality of B-mode image planes, such as in a biplane or triplane image acquisition as described in more detail herein.

The data 540-552 may be stored in the memory 560, for example, as sets of vector data values, where each set defines an individual ultrasound image frame. The vector data values are generally organized based on the polar coordinate system. Alternately or additionally the data may be stored as beamformed IQ data in the memory 204.

A scan converter sub-module 570 accesses and obtains from the memory 560 the vector data values associated with an image frame and converts the set of vector data values to Cartesian coordinates to generate an ultrasound image frames 572 formatted for display. The ultrasound image frames 572 generated by the scan converter module 570 may be provided back to the memory 560 for subsequent processing or may be provided to the memory 204.

Once the scan converter sub-module 570 generates the ultrasound image frames 572 associated with, for example, B-mode image data, and the like, the image frames 572 may be restored in the memory 560 or communicated over a bus 574 to a database (not shown), the memory 560, the memory 204, and/or to other processors.

The scan converted data may be converted into an X, Y format for video display to produce ultrasound image frames. The scan converted ultrasound image frames are provided to a display controller (not shown) that may include a video processor that maps the video to a grey-scale mapping for video display. The grey-scale map may represent a transfer function of the raw image data to displayed grey levels. Once the video data is mapped to the grey-scale values, the display controller controls the touchscreen display 120 (shown in FIG. 2) to display the image frame within the activity window 402. The image displayed within the activity window 402 may be produced from image frames of data in which each datum indicates the intensity or brightness of a respective pixel in the display.

Referring again to FIG. 5, a 2D video processor sub-module 580 combines one or more of the frames generated from the different types of ultrasound information. For example, the 2D video processor sub-module 580 may combine a different image frames by mapping one type of data to a grey map and mapping the other type of data to a color map for video display. In the final displayed 2D ultrasound image, color pixel data may be superimposed on the grey scale pixel data to form a single multi-mode image frame 582 (e.g., functional image) that is again re-stored in the memory 560 or communicated over the bus 574. Successive frames of 2D ultrasound images may be stored as a cine loop in the memory 260 or the memory 204. The cine loop represents a first in, first out circular image buffer to capture image data that is displayed to the user. The user may freeze the cine loop by entering and/or selecting a freeze command using an interface component shown on the GUI 400 of the touchscreen display 120.

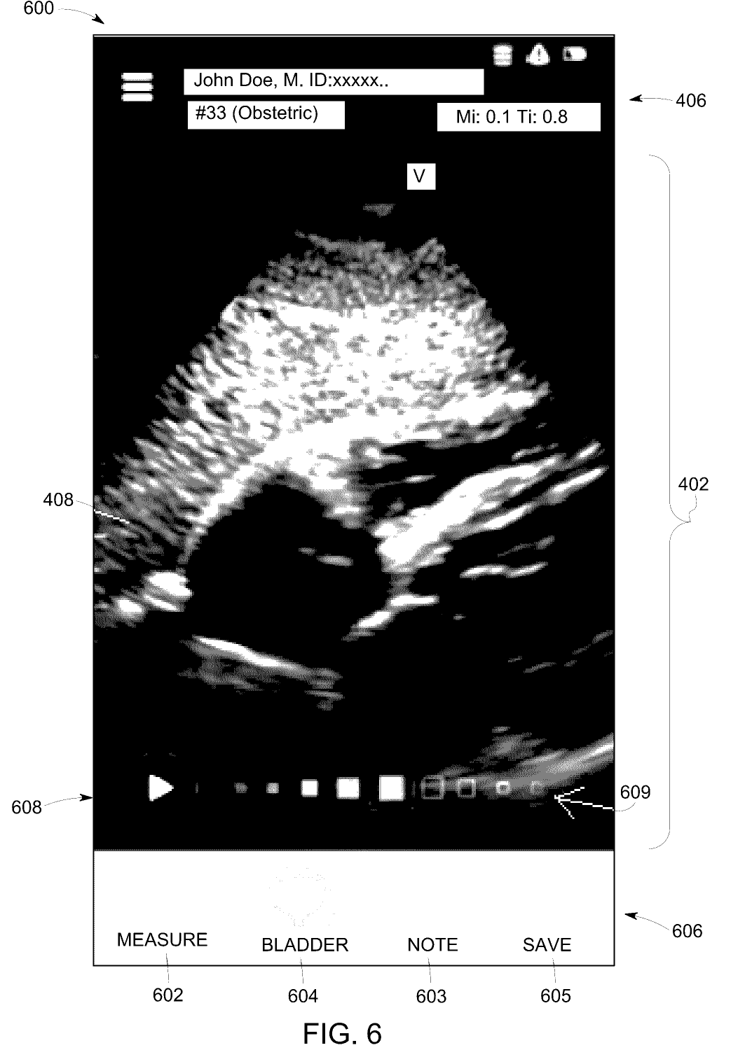

Returning to FIG. 4, the ultrasound imaging application includes diagnostic tools, which may be used on one or more 2D ultrasound images 408 acquired by the portable host system 104, as described above, or stored in the memory 204 (e.g., acquired remotely, acquired previously). For example, in connection with FIG. 6, the user may access a diagnostic and measurement tool menu 606 within the ultrasound imaging application by selecting a diagnostic and measurement icon (e.g., one of the icons 409-412).

FIG. 6 illustrates the diagnostic and measurement tool menu 606 within a GUI 600. The diagnostic and measurement tool menu 606 is shown with a plurality of icons. For example, a distance measuring icon 602, an organ volume tool icon 604, an annotation icon 603). It should be noted additional or alternative icons may be within the diagnostic and measurement tool menu 606, such as, visual diagnostic tools, in various other embodiments. It should be noted that in various other embodiments the organ volume tool icon 604 and/or user access for executing the organ volume application may correspond to one of the icons shown in FIG. 3.

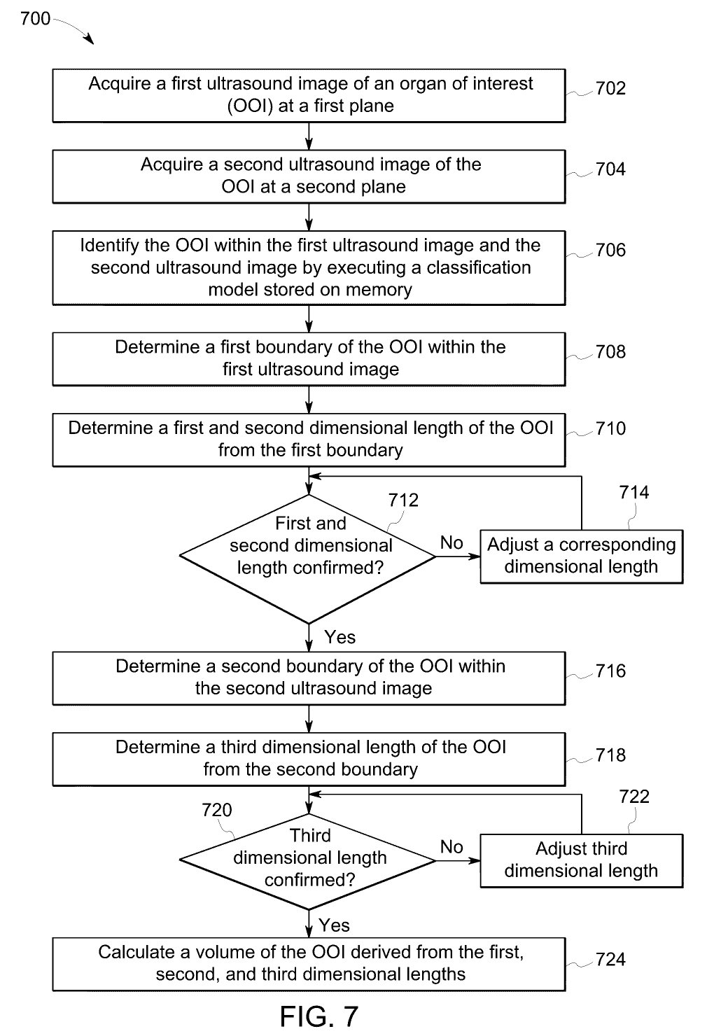

As further described in connection with a method 700 of FIG. 7, when the organ volume tool icon 604 is selected the controller 202 executes programmed instructions stored in the memory 204 corresponding to a workflow (e.g., operations of the method 700) of the organ volume application. During the executed workflow, the GUI may display one or more interface components and/or information, such as textual pop-ups or graphical guidance (e.g., arrows, animations), to assist and guide the user through the workflow (e.g., one or more operations of the method 700). For example, the controller 202 may display textual information that provides visual assistance of what information is being requested (e.g., first ultrasound image).

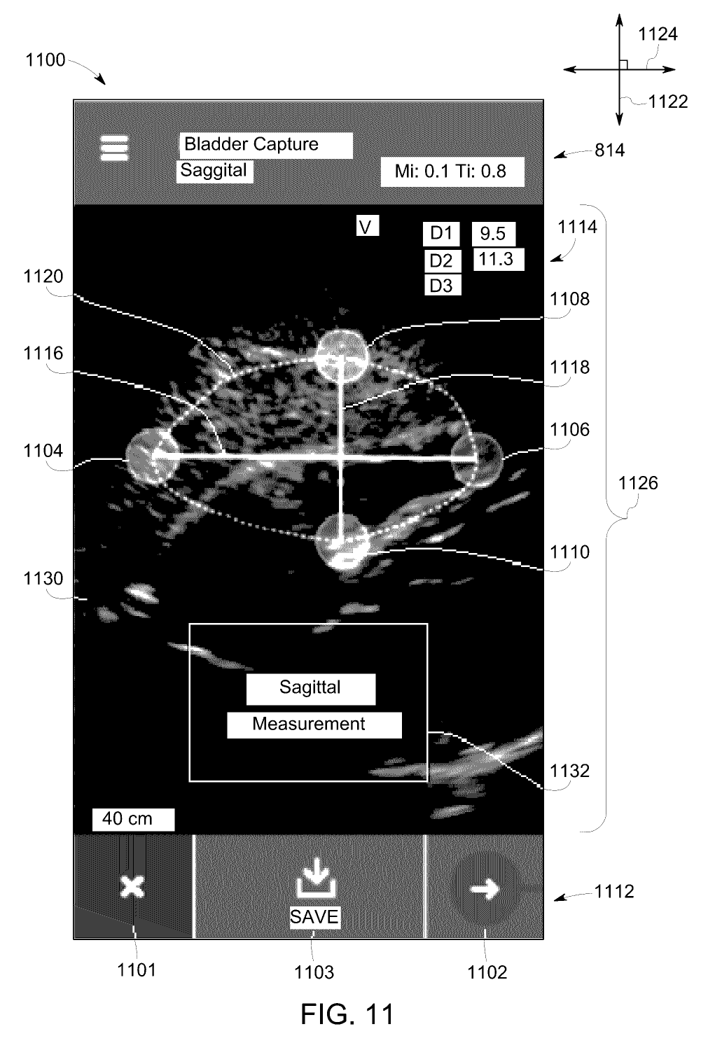

For example, the controller 202 may apply an automation algorithm on a sagittal 2D ultrasound image that includes the OOI (e.g., a bladder). The sagittal 2D ultrasound image may be selected by the user from one or more 2D ultrasound images stored in the memory 204 using a scroll bar 609 and/or acquire a new ultrasound image by selecting a graphical icon 608. The controller 202 executes a trained classification model and a contour model of the automation algorithm to the sagittal 2D ultrasound image. The contour model may be used by the controller 202 to determine the borders of the OOI (e.g., a bladder), and draws a contour or boundary (e.g., the first boundary 1120 of FIG. 11) around the OOI shown on the touchscreen display 120. The controller 202, by executing the contour model, may determine the widest orthogonal distances of the OOI corresponding to two orthogonal dimensional lengths of the OOI. The controller 202 may automatically place caliper pairs at the end points of the two dimensional lengths and calculate the distances between the corresponding caliper pairs. Optionally, the user may confirm calculated distances or adjust and/or move the calipers to improve the accuracy of the two dimensional lengths.

When the distances are confirmed, the controller 202 may automatically acquire a transverse 2D ultrasound image that is at an orthogonal plane with respect to the sagittal image. For example, the user may acquire a new ultrasound image and select the transverse image during acquisition, and/or select a transverse 2D ultrasound image stored in the memory 204.

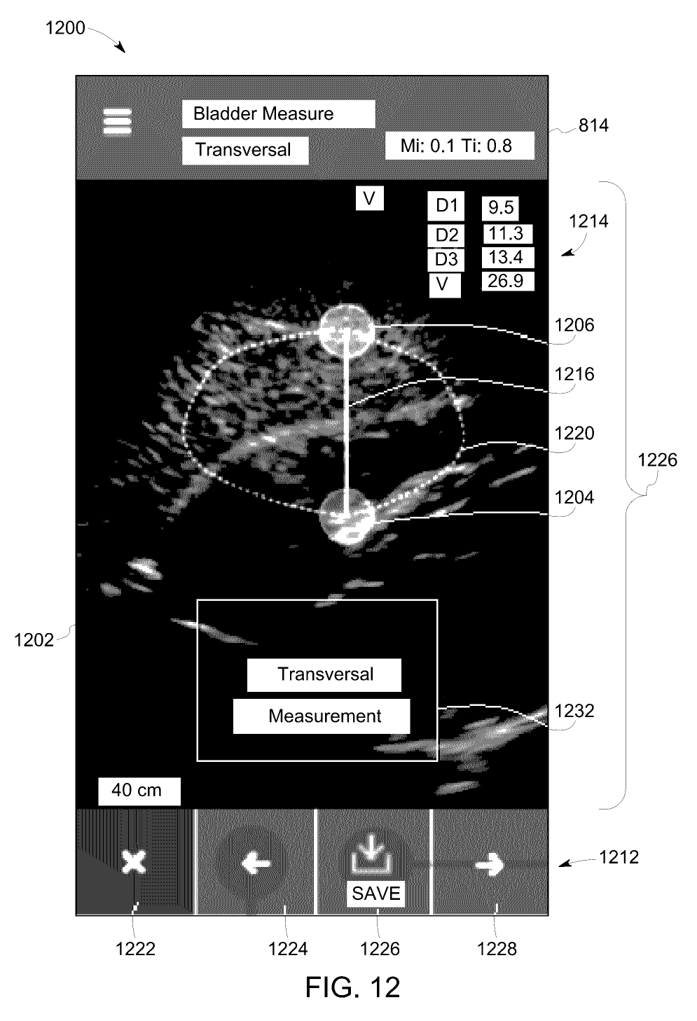

When the transverse image is selected, the controller 202 may automatically apply the automation algorithm on the transverse image. The controller 202 executes the trained classification model and the contour model of the automation algorithm to determine the boundary of the OOI (e.g., a bladder), and draws a contour or border around the OOI shown on the touchscreen display 120 (e.g., second boundary 1220 at FIG. 12). Additionally, the controller 202 by executing the contour model may determine the widest distance of the OOI, corresponding to a dimensional length of the OOI. The controller 202 may automatically place a pair of calipers at end point of the dimensional length and calculate a distance between the calipers. Optionally, the user may confirm calculated distances or adjust and/or move the calipers to improve the accuracy of the two dimensional lengths.

The controller 202 calculates a volume of the OOI based on the three distances (e.g., the two dimensional lengths from the sagittal image, the dimensional length form the transverse image) calculated from the contour model. The controller 202 may generate a report listing the corresponding distances, and display the report on the touchscreen display 120. Additionally or alternatively, the controller 202 may calculate a plurality of volumes of the OOI based on different pairs of orthogonal images (e.g., a sagittal image, a transverse image). For example, the controller 202 may calculate volumes corresponding to a Pre-Void volume and Post-Void volume of a bladder.

FIG. 7 illustrates a flowchart of the method 700 for using a mobile ultrasound system 100 to determine a volume of an organ of interest (OOI) from a plurality of 2D ultrasound images. The method 700, for example, may employ structures or aspects of various embodiments (e.g., systems and/or methods) discussed herein. In various embodiments, certain steps (or operations) may be omitted or added, certain steps may be combined, certain steps may be performed simultaneously, certain steps may be performed concurrently, certain steps may be split into multiple steps, certain steps may be performed in a different order, or certain steps or series of steps may be re-performed in an iterative fashion. In various embodiments, portions, aspects, and/or variations of the method 700 may be used as one or more algorithms or applications to direct hardware to perform one or more operations described herein. It should be noted, other methods may be used, in accordance with embodiments herein.

One or more methods may (i) acquire a first ultrasound image of an OOI at a first plane and a second ultrasound image of the OOI at a second plane; (ii) using one or more processors to identify the OOI within the first ultrasound image and the second ultrasound image by executing a classification model stored in memory; (iii) using the one or more processors to determine a first boundary of the OOI within the first ultrasound image and a second boundary of the OOI within the second ultrasound image by executing a contour model stored in memory; (iv) determine a first and second dimensional length of the OOI from the first boundary and a third dimensional length of the OOI from the second boundary; and (v) calculating a volume of the OOI from the first, second, and third dimensional lengths.

Beginning at 702 a first ultrasound image of an OOI at a first plane is acquired by the controller 202. In various embodiments, the controller 202 may acquire the first ultrasound image based on a user selection from one or more stored ultrasound images. For example, the user may slide the scroll bar 609 (FIG. 6) to display the ultrasound image in the activity area 402. When the desired first ultrasound image is displayed, the user may confirm the selection by selecting the organ volume tool icon 604 and/or selecting the save icon 605.

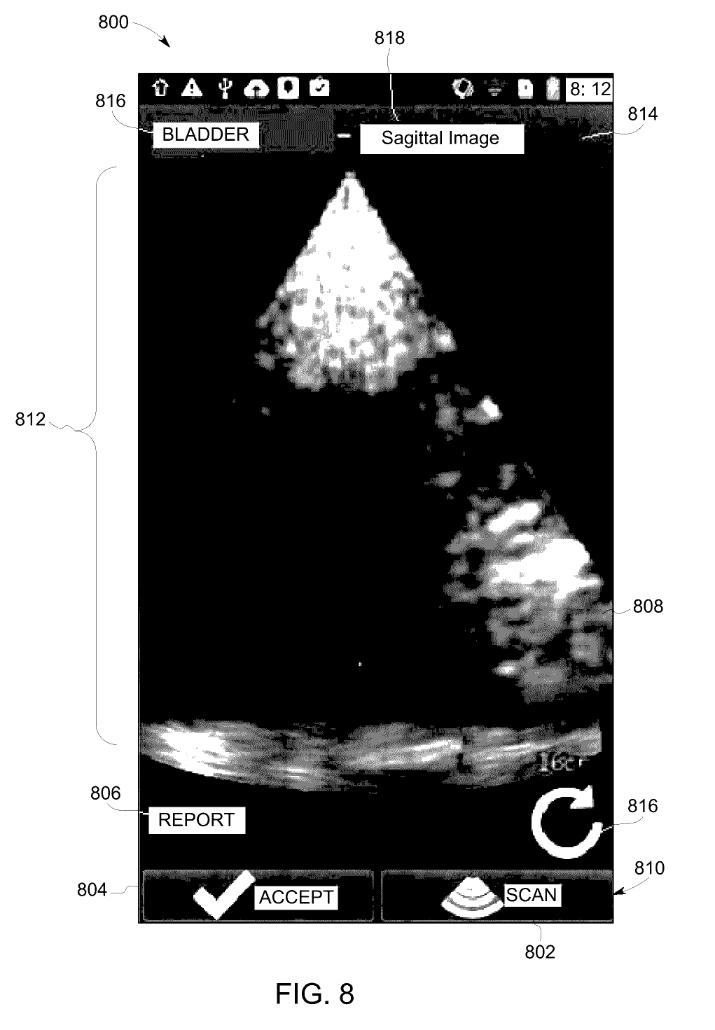

In another example, the user may select the organ volume tool icon 604. The controller 202 executes programmed instructions stored in the memory 204 corresponding to the organ volume application, which may include displaying the GUI 800 shown in FIG. 8.

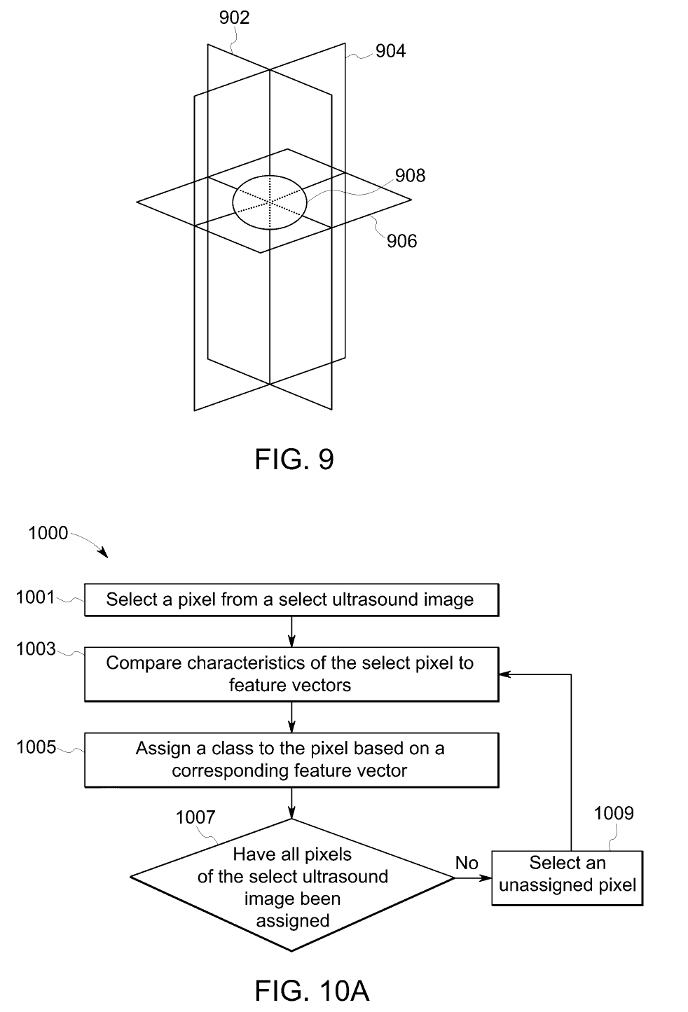

FIG. 8 illustrates the GUI 800 shown on the touchscreen display 120 to acquire the first ultrasound image. The GUI 800 may include an activity window 812, a menu bar 810, and a title bar 814. The menu bar 810 may include one or more interface components, such as a scan icon 802, a confirmation icon 804, and a stored ultrasound image icon 806 for selection and/or acquisition of the first ultrasound image. For example, when the scan icon 802 is selected, the controller 202 may establish communication with the ultrasound probe 102 via the transceiver 230 to receive ultrasound digital data acquired by the ultrasound probe 102 at a first plane 902 (FIG. 9) of the OOI, such as a sagittal plane. As described above in relation to selection of the ultrasound image acquisition icon 410 of FIG. 4, the controller 202 may process the ultrasound digital data into one or more 2D ultrasound images 808.

Optionally, an icon 816 may be displayed on the GUI 800 by the controller 202 allowing a user to freeze a 2D ultrasound image displayed on the activity window 812. For example, the controller 202 may continually generate 2D ultrasound images as the ultrasound digital data is being received from the ultrasound probe 102. The controller 202 may refresh the 2D ultrasound image 808 shown in the activity window 812 of the GUI 800 when a new 2D ultrasound image is generated by the controller 202. When the user selects the icon 816, the controller 202 may freeze and/or stop refreshing the 2D ultrasound images 808 displayed on the activity screen 812. Optionally, the controller 202 may continue to generate 2D ultrasound images and store the ultrasound images in the memory 204.

When the stored ultrasound image icon 806 is selected, the controller 202 may retrieve one or more ultrasound image stored in the memory 204 acquired along the first plane 902, and display the retrieved images on the touchscreen display 120. For example, the controller 202 may display the one or more ultrasound images in the activity window 812 in a grid and/or individually with a scroll bar. Additionally or alternatively, when the stored ultrasound image icon 806 is selected, the controller 202 stores the displayed 2D ultrasound image 808 with the activity window 812 to the memory 204.

The accept icon 804 may instruct the controller 202 that the ultrasound image 808 displayed in the activity window 812 and/or an ultrasound image selected from a plurality of ultrasound image corresponds to the first ultrasound image used to calculate the volume of the OOI.

The title bar 814 may include a graphical icon 816 and/or textual information 818 to guide the user on what stage of a workflow (e.g., operation of the method 700) to calculate a volume of the OOI and/or describe data requested by the controller 202. For example, the title bar 814 includes the graphical icon 816, such as similar to the organ volume tool icon 604 of FIG. 6, representing a diagnostic and measuring tool currently being executed by the controller 202. Optionally, in connection with FIGS. 11-13B, the textual information of the title bar 814 may be adjusted and/or updated based on the stage of the workflow (e.g., operation of the method 700).

The textual information 818 describes that an ultrasound image corresponding to a sagittal image (e.g., ultrasound image acquired along a sagittal plane with respect to the OOI) is requested by the controller 202 corresponding to the operation at 702. Additionally or alternatively, the title bar 814 may include interface components (e.g., icons) to allow a user to navigate and/or go to different stages of the workflow.

Returning to FIG. 7, at 704, a second ultrasound image of the OOI at a second plane is acquired by the controller 202. For example, the second ultrasound image may be acquired and/or selected similar to the first ultrasound image at 702. In connection with FIG. 9, the second plane 906 is orthogonal to the first plane 902.

FIG. 9 is an illustration of orthogonal planes (e.g., 902-906) of an organ of interest 908. For example, each plane 902-906 is perpendicular with respect to each other. The plane 902-906 corresponding to the ultrasound image may be based on an angle of the elements 210 and 212, shown in FIG. 2, with respect to the OOI when the ultrasound probe 102 acquires the received echoes reflected from the patient. The first plane 902 may correspond to a sagittal plane, the second plane 904 may correspond to a transverse plane, and the third plane 906 may correspond to coronal plane.

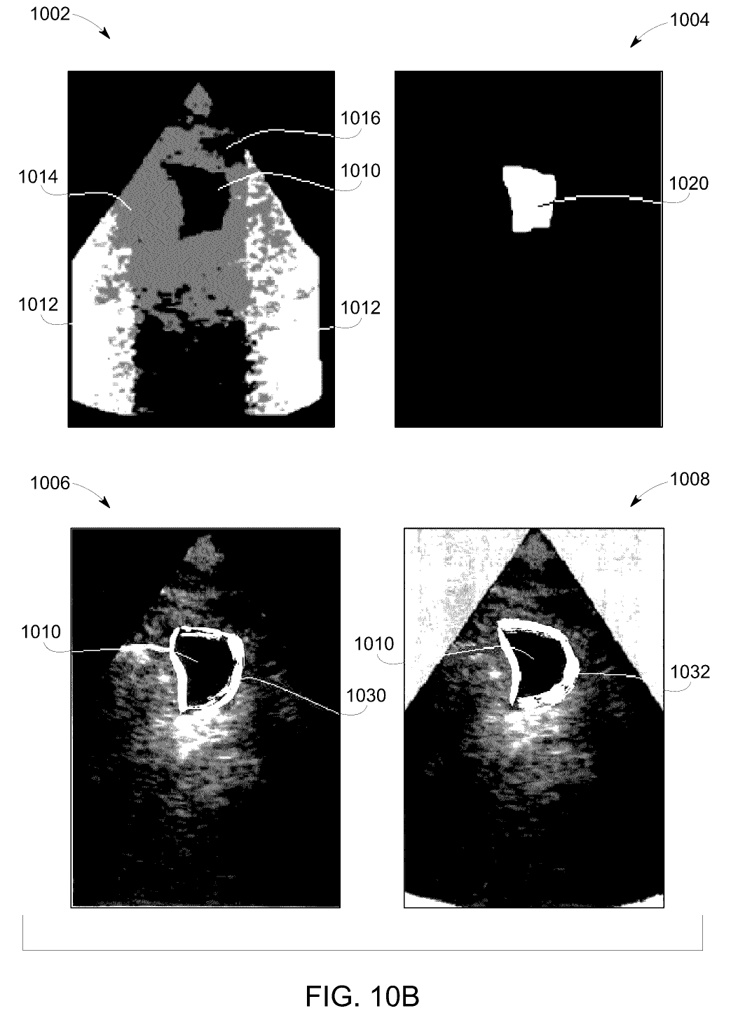

At 706, the OOI within the first ultrasound image and the second ultrasound image is identified by the controller 202 executing a classification model stored in the memory 204. The classification model may correspond to a machine learning algorithm based on a classifier (e.g., random forest classifier) that builds a pixel level classifier model to label and/or assign each pixel of the first ultrasound image and the second ultrasound image into a plurality of categories or classes (e.g., muscle, fat, background anatomy, organ of interest). The classification model may determine the classes from a feature space of the pixels based from the various intensities and spatial position of pixels of the first ultrasound image and the second ultrasound image. For example, in connection with FIG. 10, the controller 202 may determine based on the classification model, pixels of the first ultrasound image and the second ultrasound image that correspond to the OOI.

FIG. 10A illustrates a workflow diagram 1000 of the controller 202 for executing a classification model. FIG. 10B illustrates an ultrasound image being adjusting during execution of the classification model (e.g., the workflow diagram 1000) and a contour model (e.g., at 706) relating to one of the ultrasound images (e.g., the first ultrasound image, the second ultrasound image).

In various embodiments, certain steps (or operations) of the workflow diagram 1000 may be omitted or added, certain steps may be combined, certain steps may be performed simultaneously, certain steps may be performed concurrently, certain steps may be split into multiple steps, certain steps may be performed in a different order, or certain steps or series of steps may be re-performed in an iterative fashion. For example, the controller 202, by executing the classification model, assigns and/or labels the pixels of the ultrasound image into classes corresponding to portions of an anatomy of the patient. In connection with FIG. 10B, the controller 202 determines which pixels correspond to a background anatomy 1012, muscle tissue 1014, fat 1016, and/or the OOI 1010. The controller 202 may assign the pixels based on feature vectors of the classification model corresponding to one or more classes.

Returning to FIG. 10A, at 1001, the controller 202 selects a pixel from a select ultrasound image (e.g., the first ultrasound image, the second ultrasound image).

At 1003, the controller 202 compares characteristics of the select pixel to feature vectors. For example, the controller 202 may compare an intensity or brightness of the select pixel to feature vectors of the classification model. In another example, the controller 202 may determine a variance kurtosis, skewness, or spatial distribution characteristic of the select pixel by comparing the intensity of the select pixel with adjacent and/or proximate pixels around the select pixel. A number of characteristics of the select pixel compared by the controller 202 may be based on the feature sets included in the feature vectors.

Each feature vector is an n-dimensional vector that includes three or more features of pixels (e.g., mean, variance, kurtosis, skewness, spatial distribution) corresponding to a class (e.g., a background anatomy 1012, muscle tissue 1014, fat 1016, the OOI 1010) of pixels of anatomy within an ultrasound image. The feature vectors of the classification model may be generated and/or defined by the controller 202 and/or a remote system based from a plurality of reference ultrasound images.

For example, the controller 202 may select pixel blocks from one hundred reference ultrasound images. The select pixel blocks may have a length of five pixels and a width of five pixels. The select pixel blocks may be selected and/or marked by the user to correspond to one of the classes (e.g., muscle, fat, background anatomy, tissue of the OOI). For example, a plurality of pixels within each select pixel block may represent and/or correspond to one of the classes, such as tissue of the OOI. Based on the plurality of pixels within the select pixel blocks, the controller 202 may generate and/or define a feature vector.

For example, the controller 202 may determine feature sets for each pixel within the plurality of pixels of a select pixel block or more than one select pixel block corresponding to the same class. One of the feature sets may be based on an intensity histogram of the reference ultrasound images. For example, the controller 202 may calculate a mean intensity of the plurality of pixels, a variance of the plurality of pixel intensities, a kurtosis or shape of intensity distribution of the plurality of pixels, a skewness of the plurality of pixels, and/or the like. Additionally, one of the feature sets may correspond to a position or spatial feature of the pixels within the select pixel block. For example, a spatial position with respect to a position within the reference image (e.g., central location) and a depth with respect to an acquisition depth within the patient. The controller 202 may perform a k-means clustering and/or random forest classification on the feature sets to define feature values that correspond to the class of the select pixel blocks. The controller 202 may define a feature vector corresponding to the class based on the feature values to the classification model.

Additionally or alternatively, the feature vector may be further defined based on a validation analysis. For example, the controller 202 use a k-fold cross validation by subdividing the select pixel blocks with a plurality of pixels for one of the classes into k random parts with (k−1) parts being used by the controller 202 to define the feature vector and the remaining select pixel blocks for testing or validation of the feature vector.

Additionally or alternatively, the controller 202 may further assign each of the plurality of pixels binary codes (e.g., an eight digit binary code). For example, the binary code may be derived by comparing a center pixel value of the select pixel block with the remaining plurality of pixels within the select pixel block.

At 1005, the controller 202 may assign a class to the select pixel based on a corresponding feature vector. For example, the controller 202 may determine a candidate feature vector that includes feature sets that are approximately the same and/or within a set threshold to the characteristics of the select pixel based on the comparison at 1003. The controller 202 may assign the class of the candidate feature vector to the select pixel. For example, as shown in 1002 in FIG. 10B, the controller 202 may assign a background anatomy 1012, muscle tissue 1014, fat 1016, and/or the OOI 1010 class to the select pixel.

When the select pixel is assigned a class, the controller 202 may repeat the classification model to the remaining pixels of the select ultrasound image, as shown at 1007 and 1009 of FIG. 10A.

Optionally, as shown at 1004 of FIG. 10B, the controller 202 may perform a binary mask to partition or isolate pixels corresponding to the OOI 1020 from the ultrasound image. For example, the controller 202 may execute the classification model stored in the memory 102 to identify the plurality of pixels corresponding to the OOI from the ultrasound image. The controller 202 may generate a binary mask based on the identified pixels of the OOI and the remaining pixels of the ultrasound image. The controller 202 utilizing the binary mask on the ultrasound image, may extract the pixels corresponding to the OOI from the ultrasound image.

Returning to FIG. 7, at 708 a first boundary of the OOI within the first ultrasound image is determined by the controller 202. In connection with FIG. 10, the controller 202 may determine a boundary 1032 of the OOI by executing a contour model stored in the memory 204 to determine the first boundary of the OOI.

As shown at 1006 of FIG. 10B, the controller 202 may form an initial boundary 1030 of the OOI. The initial boundary 1030 may be determined based on the identified pixels determined from the classification model and/or the binary mask at 1004.

As shown at 1008 of FIG. 10B, the controller 202 may adjust the initial boundary 1030 by executing the contour model stored in the memory 204 to form the boundary 1032. The contour model may be based on traditional active contour models (e.g., snakes) with an additional distance regularization term to intrinsically maintain the regularity of a zero level set, the variable φ of Equation 1, while the controller 202 executes the contour model. The distance regularization term may use a double well potential function such that the derived level set evolution has a unique forward and backward diffusion effects. The distance regularization term e issues the address of curve re-initialization and maintains the shape of evolving front. Additionally, the contour model may include an external energy term defined by equation 1. The external energy term may be based on image gradients, shown as the variable F, to drive a motion of the level curve to desired locations corresponding to the boundary of the OOI. The variable A of Equation 1 correspond to an area of the OOI, and the variable dₚ is the potential function.

$$\frac{\partial φ}{\partial t} = μ div ( {d}_{p} ( \vert ∇ φ \vert ) ∇ φ ) + F \vert ∇ φ \vert + A \cdot ∇ φ \qquad Equation 1$$

Returning to FIG. 7, at 710 the controller 202 determines a first and second dimensional length 1116-1118 of the OOI from a first boundary 1120.

FIG. 11 illustrates a GUI 1100 shown on the touchscreen display 120. The GUI 1100 includes an activity window 1126 that shows a first ultrasound image 1130 of the OOI outlined by a first boundary 1120. The GUI 1100 displays the first dimensional length 1116 and the second dimensional length 1118 overlaid on the OOI. The first dimensional length 1116 and the second dimensional length 1118 correspond to two orthogonal or perpendicular lengths of the OOI formed from pairs of opposing points along the first boundary 1120. The first and second dimensional lengths 1116 and 1118 may correspond to dimensions of the OOI, such as a height and width. The GUI 1100 further may include opposing calipers 1104-1106 and 1108-1110 on the first boundary 1120 defining the first dimensional length 1116 and the second dimensional length 1118, respectively.

For example, the first dimensional length 1116 is formed from opposing points shown by the calipers 1104 and 1106. The calipers 1104 and 1106 are positioned at a widest point or distance of the first boundary 1120 along an axis 1124. Thereby, the first dimensional length 1116 is parallel to the axis 1124. The widest point of the first dimensional length 1116 corresponding to a distance between the opposing points of the first boundary 1120 having a greater length than other opposing points along the first boundary 1120 that are parallel to the axis 1124.

In another example, the second dimensional length 1118 is formed from opposing points shown by the calipers 1108 and 1110. The calipers 1108 and 1110 are positioned at a widest point or distance of the first boundary 1120 along an axis 1122. Thereby, the second dimensional length 1118 is parallel to the axis 1122. The widest point of the second dimensional length 1118 corresponding to a distance between the opposing points of the first boundary 1120 having a greater length than other opposing points of the first boundary 1120 that are parallel to the axis 1122.

It should be noted that axes 1120-1122 are illustrative and may have a different orientation (e.g., rotated, at a different angle) with respect to various other embodiments. Optionally, the controller 202 may rotate the axes 1120-1122 when determining the first and second dimensional length 1116-1118. For example, the controller 202 may rotate the axes 1120-1122 to confirm the first and second dimensional lengths 1116-1118 correspond to two dimensions of the OOI by having a greater length relative to other rotational angles of the axes 1120-1122.

Optionally, the controller 202 may instruct the touchscreen display 120 to show a numerical representation of the first dimensional length 1116 (e.g., labeled as D1) and the second dimensional length 1118 (e.g., labeled as D2) within a measurement window 1114 overlaid on the GUI 1100.

In various embodiments, the GUI 1100 may include one or more interface components and/or information, such as the textual pop-up window 1132 or graphical guidance (e.g., arrows, animations), to assist and guide the user through the workflow (e.g., one or more operations of the method 700). For example, the textual pop-up window 1132 is shown displaying textual information that provides visual assistance or guidance information on what is being acquired (e.g., first and second dimensional lengths 1116-1118) and/or what stage of the workflow the GUI 1100 represents.

At 712, the controller 202 may determine whether the first and second dimensional lengths 1116-1118 are confirmed. For example, the controller 202 automatically determines the first and second dimensional lengths 1116-1118. The GUI 1100 may include a menu bar 1112 with one or more interface components, such as icons 1101-1103 that may aid in guiding the user through the workflow (e.g., one or more operations of the method 700) and/or confirming the first and second dimensional lengths 1116-1118. For example, the icons 1101-1102 may allow a user to move (e.g., advance to a subsequent operation) through the workflow associated with the method 700.

For example, when the icon 1101 is selected the controller 202 may exit the workflow allowing the user to exit the organ volume tool. In another example, when the icon 1102 is selected the controller 202 may confirm the first and second dimensional length 1116-1118 and proceed to the next step in the workflow (e.g., 716). Optionally, the controller 202 may overlay a popup window when the first and second dimensional lengths 1116-1118 are confirmed to notify the user.

Optionally, the GUI 1100 may include a save icon 1103. When the save icon 1103 is selected by the user, the controller 202 may capture and/or save the first ultrasound image 1130 in the memory 204.

Optionally, at 714, the first dimensional length 1116 and/or the second dimensional length 1118 may be adjusted by the controller 202. For example, a user via the touchscreen display 120 may select and reposition one of the calipers 1104 or 1106. The controller 202 may reposition the selected caliper 1104 or 1106 displayed on the GUI 1100 in response to the change in position of the selected caliper 1104 or 1106 by the user. The controller 202 updates and/or adjusts the first dimensional length 1116 based on the repositioned opposing caliper 1104 or 1106. Additionally or alternatively, the controller 202 may update the measurement window 1114 based on the adjusted first dimensional length 1116.

In another example, a user via the touchscreen display 120 may select and reposition one of the calipers 1108 or 1110. The controller 202 may reposition the selected caliper 1108 or 1110 displayed on the GUI 1100 in response to the change in position of the selected caliper 1108 or 1110 by the user. The controller 202 updates and/or adjusts the second dimensional length 1118 based on the repositioned opposing calipers 1108 and 1110.

Additionally or alternatively, the controller 202 may update the feature vector of the classification model based on pixels at and/or proximate to the repositioned calipers 1104-1110. For example, the caliper 1104 was repositioned based on a position received from a user via the touchscreen display 120. The controller 202 may determine a plurality of pixel features (e.g., mean pixel intensity, a pixel variance, a skewness, spatial position, kurtosis) from pixels located at and/or around the adjust position of the caliper 1104, and update the feature vector based on the plurality of pixel features.

Returning to FIG. 7, when the controller 202 determines at 712 that the first and second dimensional lengths 1116-1118 are confirmed, at 716, the controller 202 may determine a second boundary of the OOI with the second ultrasound image. For example, similar to 708, the controller 202 may determine a second boundary of the OOI executing a contour model stored in the memory 204 to determine the second boundary of the OOI.

FIG. 12 illustrates a GUI 1200 shown on the touchscreen display 120. The GUI 1200 includes an activity window 1226 that shows a second ultrasound image 1202 of the OOI. The second ultrasound image 1202 is outlined by a second boundary 1220 determined by the controller 202.

At 718, the controller 202 determines a third dimensional length 1216 of the OOI from the second boundary 1220. The third dimensional length 1216 may be formed from two opposing points along the second boundary 1220. The third dimensional length 1216 may correspond to dimensions of the OOI, such as a height. The GUI 1200 may further include opposing calipers 1204 and 1206 that define the third dimensional length 1216. For example, the third dimensional length 1116 is formed from opposing points shown by the calipers 1204 and 1206. The calipers 1204 and 1206 are positioned at a widest point or distance along the second boundary 1220 to form the third dimensional length 1216. The widest point of the third dimensional length 1216 corresponding to a distance between the opposing points of the second boundary 1220 having a greater length than other opposing points along the second boundary 1220.

Optionally, the controller 202 may instruct the touchscreen display 120 to show a numerical representation of the first dimensional length 1116 (e.g., labeled as D1), the second dimensional length 1118 (e.g., labeled as D2), and the third dimensional length 1216 (e.g., labeled as D3) within a measurement window 1214 overlaid on the GUI 1200. Additionally or alternatively, the controller 202 may further display a volume (e.g., labeled as V), determined at 724 of the method 700 within the measurement window 1214.

In various embodiments, the GUI 1200 may include one or more interface components and/or information, such as the textual pop-up window 1232 or graphical guidance (e.g., arrows, animations), to assist and guide the user through the workflow (e.g., one or more operations of the method 700). For example, the textual pop-up window 1232 is shown displaying textual information that provides visual assistance of what information is being acquired (e.g., third dimensional length 1216) and/or what stage of the workflow the GUI 1200 represents.

At 720, the controller 202 may determine whether the third dimensional length 1216 is confirmed. For example, the controller 202 automatically determines the third dimensional length 1216. The GUI 1200 may include a menu bar 1212 with one or more interface components, such as icons 1222-1228 that may aid in guiding the user through the workflow (e.g., one or more operation of the method 700) and/or confirming the third dimensional length 1216. For example, the icons 1222-1228 may allow a user to move (e.g., advance to a subsequent operation of the method 700, return to a previous operation of the method 700) through the workflow associated with the method 700.

For example, when the icon 1222 is selected the controller 202 may exit and/or cancel the workflow allowing the user to exit the organ volume tool. In another example, the icon 1224 may instruct the controller 202 to return to a previous operation, for example return to 710 and display the GUI 1100. In another example, when the icon 1228 is selected the controller 202 may confirm the third dimensional length 1216 and proceed to the next step in the workflow, for example display the GUI 1300, 1301. Optionally, the controller 202 may overlay a popup window when the third dimensional length 1216 is confirmed to notify the user.

In various embodiments, the GUI 1200 may include a save icon 1226. When the save icon 1226 is selected by the user, the controller 202 may capture and/or save the second ultrasound image 1220 in the memory 204.

Optionally, at 722 the third dimensional length 1216 may be adjusted by the controller 202. For example, a user via the touchscreen display 120 may select and reposition one of the calipers 1204 or 1206. The controller 202 may reposition the selected caliper 1204 or 1206 displayed on the GUI 1200 in response to the change in position of the selected caliper 1204 or 1206 by the user. Based on the repositioned opposing caliper 1204 or 1206, the controller 202 updates and/or adjusts the first dimensional length 1216 formed by the opposing calipers 1204-1206. Additionally or alternatively, the controller 202 may update the measurement window 1214 based on the adjusted third dimensional length 1216. Optionally, the controller 202 may update the feature vector of the classification model based on pixels at and/or proximate to the repositioned caliper 1204-1206.

When the controller 202 determines at 720 that the third dimensional length 1216 is confirmed, at 724, the controller 202 may calculate a volume of the OOI derived from the first, second, and third dimensional lengths 1116, 1118, and 1216. For example, the controller 202 may calculate the volume of the OOI based on Equation 2 for determining a volume (v) corresponding to an OOI having a shape of approximately an ellipsoid. For example, the variable a may represent the first dimensional length 1116, the variable b may represent the second dimensional length 1118, and the variable c may represent the third dimensional length 1216.

v=0.52·a·b·c Equation 2:

It should be noted that in various embodiments the controller 202 may determine the volume of the OOI prior to confirmation of the third dimensional length 1216 at 720. For example, the controller 202 may calculate the volume of the OOI based on the third dimensional length 1216 determined at 718 and display the calculated volume within the measurement window 1214 of FIG. 12.

It should be noted, that the method 700 may be repeated. For example, the controller 202 may obtain two sets of first, second, and third dimensional measurements 1116, 1118, and 1216 to compare differences in volumes of the OOI.

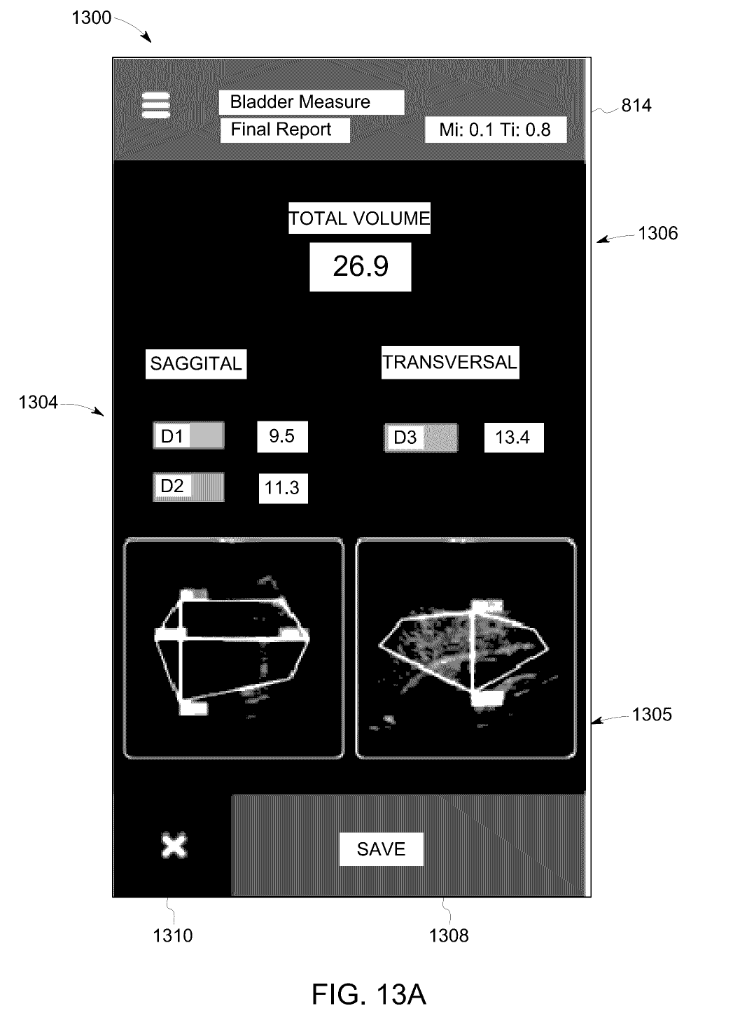

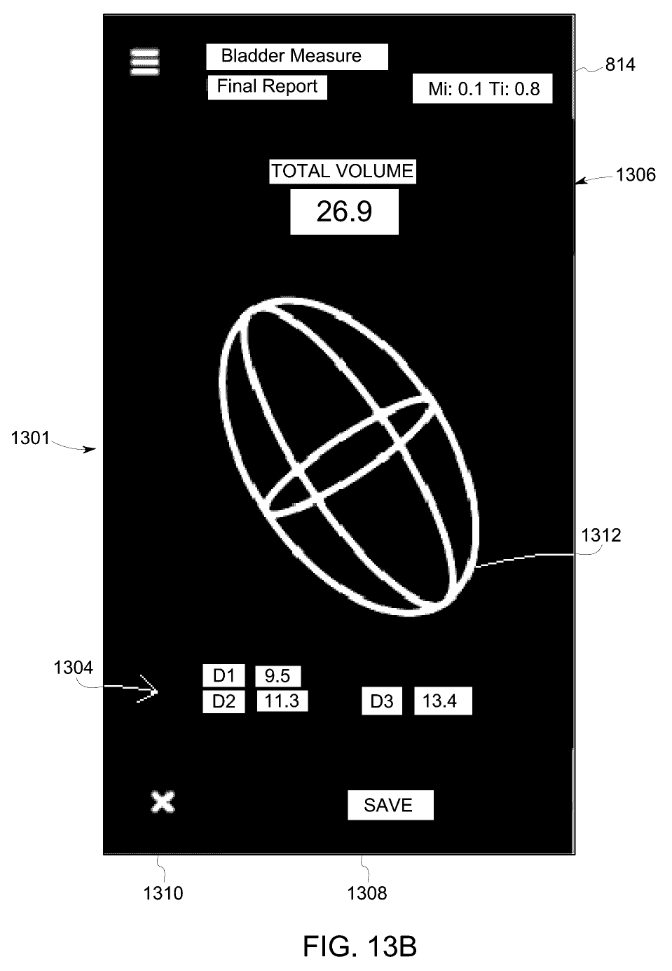

FIGS. 13A-B illustrate GUIs 1300-1301 that may be shown on the touchscreen display 120 by the controller 202, for example, when the icon 1228 is selected. Optionally, the GUIs 1300-1301 may display patient information, which may be edited and/or selected by the user via the touchscreen display 120. The GUIs 1300-1301 display a measurement window 1304 that includes numerical values representing the first dimensional length 1116 (e.g., labeled D1) and the second dimensional length 1118 (e.g., labeled D2) of the first ultrasound image 1130 (e.g., the sagittal image), and the third dimensional length 1216 (e.g., labeled D3) of the second ultrasound image 1202. The GUIs 1300-1301 may further display a volume window 1306 that includes a numerical value representing the calculated volume of the OOI determined at 724.

The GUIs 1300-1301 may further display one or more interface components, such as the icons 1308-1310. For example, when the icon 1308 is selected, the controller 202 may save the measurements displayed in the measurement window 1304 and the volume window 1306 to the memory 204. Optionally, when the icon 1308 is selected, the controller 202 may transmit the measurements displayed in the measurement window 1304 and the volume window 1306 to a remote location (e.g., a server, another device, printer). In another example, when the icon 1310 is selected the controller 202 may exit and/or cancel the workflow allowing the user to exit the organ volume tool.

Optionally, the GUI 1300 may further display screenshots 1305 of the first ultrasound image 1130 and the second ultrasound image 1202 overlaid with corresponding measurements (e.g., the first dimensional length 1116, the second dimensional length 1118, the third dimensional length 1216, the first boundary 1120, the second boundary 1220) concurrently with the measurement window 1304.

Optionally, the GUI 1301 may further display a rendering 1312 of the OOI based on the first dimensional length measurement 1116, the second dimensional length measurement 1118, and the third dimensional length measurement 1216.

Additionally or alternatively, the GUIs 1300-1301 may concurrently display more than two measurement windows 1304. For example, the GUIs 1300-1301 may display a first and second measurement window to allow a user to compare measurements of the OOI at two different times and/or ultrasound images. The first measurement window may include a first dimensional length (e.g., labeled D1), a second dimensional length (e.g., labeled D2), and a third dimensional length of a Pre-Void volume of a bladder (e.g., the OOI) determined from a first ultrasound image and a second ultrasound image. The second measurement window may include a first dimensional length (e.g., labeled D1), a second dimensional length (e.g., labeled D2), and a third dimensional length of a Post-Void volume of the bladder (e.g., the OOI) from a third ultrasound image and a fourth ultrasound image.

It should be noted that the various embodiments may be implemented in hardware, software or a combination thereof. The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a solid-state drive, optical disk drive, and the like. The storage device may also be other similar means for loading computer programs or other instructions into the computer or processor.

As used herein, the term “computer,” “subsystem” or “module” may include any processor-based or microprocessor-based system including systems using microcontrollers, reduced instruction set computers (RISC), ASICs, logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments. The set of instructions may be in the form of a software program. The software may be in various forms such as system software or application software and which may be embodied as a tangible and non-transitory computer readable medium. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, a structure, limitation, or element that is “configured to” perform a task or operation is particularly structurally formed, constructed, or adapted in a manner corresponding to the task or operation. For purposes of clarity and the avoidance of doubt, an object that is merely capable of being modified to perform the task or operation is not “configured to” perform the task or operation as used herein. Instead, the use of “configured to” as used herein denotes structural adaptations or characteristics, and denotes structural requirements of any structure, limitation, or element that is described as being “configured to” perform the task or operation. For example, a controller circuit, processor, or computer that is “configured to” perform a task or operation may be understood as being particularly structured to perform the task or operation (e.g., having one or more programs or instructions stored thereon or used in conjunction therewith tailored or intended to perform the task or operation, and/or having an arrangement of processing circuitry tailored or intended to perform the task or operation). For the purposes of clarity and the avoidance of doubt, a general purpose computer (which may become “configured to” perform the task or operation if appropriately programmed) is not “configured to” perform a task or operation unless or until specifically programmed or structurally modified to perform the task or operation.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments without departing from their scope. While the dimensions and types of materials described herein are intended to define the parameters of the various embodiments, they are by no means limiting and are merely exemplary. Many other embodiments will be apparent to those of skill in the art upon reviewing the above description. The scope of the various embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112(f) unless and until such claim limitations expressly use the phrase “means for” followed by a statement of function void of further structure.

This written description uses examples to disclose the various embodiments, including the best mode, and also to enable any person skilled in the art to practice the various embodiments, including making and using any devices or systems and performing any incorporated methods. The patentable scope of the various embodiments is defined by the claims, and may include other examples that occur to those skilled in the art. Such other examples are intended to be within the scope of the claims if the examples have structural elements that do not differ from the literal language of the claims, or the examples include equivalent structural elements with insubstantial differences from the literal language of the claims.

What is claimed is:

1. A method of using a mobile ultrasound imaging system to determine a volume of an organ of interest (OOI) from a plurality of ultrasound images comprising:
acquiring a first ultrasound image of an OOI at a first plane and a second ultrasound image of the OOI at a second plane, wherein the first plane is orthogonal to the second plane;
using one or more processors to identify the OOI within the first ultrasound image and the second ultrasound image by executing a classification model stored in memory, the classification model having a feature vector corresponding to the OOI;
using the one or more processors to determine a first boundary of the OOI within the first ultrasound image and a second boundary of the OOI within the second ultrasound image by executing a contour model stored in the memory;
determining first and second dimensional lengths of the OOI from the first boundary and a third dimensional length of the OOI from the second boundary; and
calculating a volume of the OOI derived from the first, second, and third dimensional lengths.

2. The method of claim 1, further comprising:
selecting pixel blocks from a plurality of reference ultrasound images of the OOI, wherein the pixel blocks include a plurality of pixels representing a portion of the OOI; and
generating the feature vector based from a plurality of pixel features of the pixel blocks.

3. The method of claim 2, wherein the pixel features of the pixel blocks includes at least one of a mean pixel intensity, a pixel variance, a skewness, spatial position or kurtosis.

4. The method of claim 1, further comprising adjusting at least one of a first dimensional length, a second dimensional length, or a third dimensional length based on a position received from a graphical user interface.

5. The method of claim 4, further comprising:
determining a plurality of pixel features from pixels located at the position; and
update the feature vector based on the plurality of pixel features.

6. The method of claim 1, further comprising:
displaying the first ultrasound image with the first boundary on a display; and
overlaying opposing first and second calipers on the first boundary corresponding to the first dimension of the OOI and opposing third and fourth calipers on the first boundary corresponding to the second dimension of the OOI.

7. The method of claim 6, further comprising:
adjusting a position of at least one of the first caliper, the second caliper, the third caliper, or the fourth caliper based on an input received from a graphical user interface; and
updating one of a corresponding first dimension or second dimension of the OOI based on the adjusted position.

8. The method of claim 1, further comprising displaying a textual pop-up window concurrently with the first ultrasound image, wherein the textual pop-up window includes guidance information.

9. The method of claim 1, wherein the first ultrasound image and the second ultrasound image are acquired using an ultrasound probe in communication with a mobile ultrasound imaging system, the mobile ultrasound imaging system being at least one of a mobile phone or a tablet computer.

10. The method of claim 1, wherein the OOI is a bladder.

11. A mobile ultrasound imaging system comprising:
a portable host system having one or more processors and a memory for storing a plurality of applications that include corresponding programmed instructions, wherein when a select application is activated the one or more processors execute programmed instructions of the select application by performing the following operations:
acquire a first ultrasound image of an organ of interest (OOI) at a first plane and a second ultrasound image of the OOI at a second plane, wherein the first plane is orthogonal to the second plane;
identify the OOI within the first ultrasound image and the second ultrasound image by executing a classification model stored in memory, the classification model having a feature vector corresponding to the OOI;
determine a first boundary of the OOI within the first ultrasound image and a second boundary of the OOI within the second ultrasound image by executing a contour model stored in the memory;
determine first and second dimensional lengths of the OOI from the first boundary and a third dimensional length of the OOI from the second boundary; and
calculate a volume of the OOI derived from the first, second, and third dimensional lengths.

12. The mobile ultrasound imaging system of claim 11, wherein the portable host system includes a display displaying the first ultrasound image with the first boundary and opposing first and second calipers on the first boundary corresponding to the first dimension of the OOI and opposing third and fourth calipers on the first boundary corresponding to the second dimension of the OOI.

13. The mobile ultrasound imaging system of claim 12, wherein the display further displays a graphical user interface,
the one or more processors further adjust a position of at least one of the first caliper, the second caliper, the third caliper, or the fourth caliper based on an input received from the graphical user interface and update one of a corresponding first dimension or second dimension of the OOI based on the adjusted position.

14. The mobile ultrasound imaging system of claim 13, wherein the one or more processors further determine a plurality of pixel features from pixels located at the adjusted position and update the feature vector based on the plurality of pixel features.

15. The mobile ultrasound imaging system of claim 14, wherein the pixel features include at least one of a mean pixel intensity, a pixel variance, a skewness, spatial position or kurtosis.

16. The mobile ultrasound imaging system of claim 11, further comprising an ultrasound probe having a transducer array for acquiring ultrasound data, the ultrasound probe communicatively coupled to the portable host, wherein the one or more processors acquire the first ultrasound image and the second ultrasound image from the ultrasound data acquired by the ultrasound probe.

17. The mobile ultrasound imaging system of claim 11, wherein the OOI is a bladder.

18. A tangible and non-transitory computer readable medium comprising one or more programmed instructions configured to direct one or more processors to:
acquire a first ultrasound image of an OOI at a first plane and a second ultrasound image of the OOI at a second plane, wherein the first plane is orthogonal to the second plane;
identify the OOI within the first ultrasound image and the second ultrasound image by executing a classification model stored in memory, the classification model having a feature vector corresponding to the OOI;
determine a first boundary of the OOI within the first ultrasound image and a second boundary of the OOI within the second ultrasound image by executing a contour model stored in the memory;
determine first and second dimensional lengths of the OOI from the first boundary and a third dimensional length of the OOI from the second boundary; and
calculate a volume of the OOI derived from the first, second, and third dimensional lengths.

19. The tangible and non-transitory computer readable medium of claim 18, wherein the one or more processors are further directed to:
select pixel blocks from a plurality of reference ultrasound images of the OOI, wherein the pixel blocks include a plurality of pixels representing a portion of the OOI; and
generate the feature vector based from a plurality of pixel features of the pixel blocks.

20. The tangible and non-transitory computer readable medium of claim 18, wherein the one or more processors are further directed to adjust at least one of a first dimensional length, a second dimensional length, or a third dimensional length based on a position received from a graphical user interface.
